(19) United States
(12) Patent Application Publication
(10) Pub. No.: US 2016/0287071 A1
(43) Pub. Date: Oct. 6, 2016

(54) METHODS OF MEASURING TOTAL RETINAL BLOOD FLOW USING EN FACE DOPPLER OCT

(71) Applicants: Ou Tan, Portland, OR (US); David Huang, Portland, OR (US)

(72) Inventors: Ou Tan, Portland, OR (US); David Huang, Portland, OR (US)

(73) Assignee: OREGON HEALTH & SCIENCE UNIVERSITY, PORTLAND, OR (US)

(21) Appl. No.: 15/089,803

(22) Filed: Apr. 4, 2016

Related U.S. Application Data
(60) Provisional application No. 62/142,941, filed on Apr. 3, 2015.

(51) Int. Cl. A61B 3/12; G06T 5/00; G06T 7/00; A61B 3/10; A61B 3/00

(52) U.S. Cl. CPC A61B 3/1233; A61B 3/102; A61B 3/0025; G06T 7/0012; G06T 5/002; G06T 2207/30041; G06T 2207/30104; G06T 2207/10101

(57) ABSTRACT

Methods of performing en face Doppler total retinal blood flow measurement are disclosed. The methods involve obtaining a set of structural OCT scans; calculating a noise-reduced set of Doppler phase shift images by dividing the full spectrum OCT fringe data into a set of narrower filtered frequency bands for phase shift calculation; removing bulk motion along a depth direction; detecting retinal vessels in the set of images; applying a phase unwrapping algorithm to the images; classifying the vessels as veins and arteries; and calculating total retinal blood flow based on that classification.

### ACKNOWLEDGEMENT OF GOVERNMENT SUPPORT

This invention was made with the support of the United States government under the terms of Grant Numbers R01 EY023285, DP3 DK104397, R01 EY02544, TR000128, and R01 EY013516 awarded by the National Institutes of Health. The United States government has certain rights in this invention.

### FIELD

The field generally involves the use of optical coherence tomography to measure blood flow in the eye. More specifically, the field involves the measurement of total retinal blood flow.

### BACKGROUND

Retinal blood flow is an important clinical parameter for the assessment of many ocular diseases such as age-related macular degeneration, diabetic retinopathy, glaucoma, and retinal vein occlusions (1-8). Many techniques have been used to assess ocular circulation, such as laser Doppler flowmeter, fluorescein and indocyanine green angiographies; ultrasound color Doppler imaging; magnetic resonance imaging and Doppler optical coherence tomography (OCT) (9-19). Doppler OCT is of high clinical interest due to the widespread availability of OCT instrumentation in eye clinics.

Doppler OCT detects Doppler shifts generated by light scattering particles. The Doppler shift is proportional to the axial component (parallel to the axis of beam propagation) of the velocity vector. In order to integrate flow in a blood vessel sectioned on an OCT B-scan, the angle between the OCT beam and the direction of blood flow is needed. This angle could be obtained by obtaining 2 or more cross-sectional images to establish the vessel orientation (20-28), but this introduces additional measurement error associated with the angle determination. Alternatively, a multi-beam OCT with a known angular offset between beams could be used (28-33), but this requires special multi-beam optics not available on commercial OCT instruments. To obviate the need for angle determination, one can employ high-speed 3D volumetric OCT and then integrate flow across an en face section of the blood vessel. The simplest en face approach would measure the central retinal veins and artery in the optic nerve head (ONH). However, these vessels have very high axial velocity and the Doppler shift would exceed the multiple phase wrapping limit (Doppler phase shift of more than 2π between consecutive axial scans) unless very-high-speed (200 kHz or higher) OCT systems are employed (34-36).

Currently, en face Doppler OCT approaches using clinical retinal OCT systems currently on the market are unavailable. The fastest commercially available retinal OCT in the US is a 70 kHz spectral OCT system (RTVue-XR, Optovue, Inc., Fremont, Calif.). However, this system is too slow to determine the Doppler phase shift of the central retinal artery or vein due to fringe washout and phase wrapping limits (37). Therefore, new techniques that allow measurement of the Doppler phase shift of the central retinal artery or vein are necessary.

### SUMMARY

Disclosed herein is an image processing approach that finds suitable branches of the retinal vein within the ONH, where an en face Doppler OCT flow measurement can be applied without exceeding the 2π phase wrapping limit. The image processing algorithm overcomes problems with the oblique orientation and complex branching pattern of the retinal venous vascular tree. The automated algorithm outputs the total retinal blood flow (TRBF) based on analysis of volumetric flow rates in individual vein branches within the retina. In order to overcome the variation that cardiac cycle pulsation introduces to TRBF measurement, multiple volumetric scans may be obtained in an imaging session, and TRBF values calculated from each of these volumes averaged to yield an improved TRBF measurement. In an embodiment disclosed herein, the specification of scan parameters and scan size using a commercially available OCT system is disclosed, such that a consecutive series of 5 volumetric en face Doppler scans amenable to TRBF calculation may be obtained in 3.0 seconds.

Also disclosed herein are methods for improving the signal-to-noise ratio of Doppler phase shift OCT data by dividing the full spectrum fringe data into a set of narrower frequency bands of spectral data. This splitting of the spectrum is achieved by application of a filter bank to the originally acquired OCT data. Following Fourier transformation of each these narrower spectral bands of data, the set is recombined according to an equation provided herein to yield Doppler phase shift data that exhibits improved signal quality.

Also disclosed herein are tests of the disclosed algorithm in terms of repeatability and the ability to detect reduced TRBF in glaucomatous eyes, compared to age-matched normal control eyes. Measurements of TRBF of normal and glaucomatous eyes showed that the proposed algorithm had acceptable repeatability and can be used in a large clinical study and clinical practice.

An aspect of the disclosed algorithm is that it allows measurement of TRBF in an automated fashion using commercially available OCT systems which typically have slower scanning rates than specialized OCT systems dedicated to research applications. The algorithm incorporates a multiple plane en face technique that solves the problem of phase wrapping and fringe washout in the vessels near the disc border, a nonlinear calibration function to convert Doppler phase shift to blood speed, automated removal of bulk motion artifacts, automated phase unwrapping, and an automated vessel detection and vein classification scheme.

It is an object of the disclosure to provide an image processing approach that finds branches of the retinal vein within the ONH where an en face Doppler OCT flow measurement can be applied without exceeding a 2π phase wrapping limit.

It is an object of the disclosure to provide methods to overcome problems with the application of Doppler OCT in the retina that arise due to the complex branching pattern of the retinal vasculature and the range of blood flow rates that exist therein.

It is an object of the disclosure to provide methods to identify an optimized en face plane for each individual vessel branch and to calculate Doppler phase shift within the branch using OCT.

It is an object of the disclosure to describe system calibration by a flow phantom so that Doppler phase shift values can be converted to speed.

It is an object of the disclosure to provide a method to detect a vein tree rather than an arterial tree, so that variation due to cardiac pulsatility is reduced in the TRBF calculation. It is a further object of the invention to count the contribution of each vein branch within the vein tree only once in a retinal blood flow calculation.

It is an object of the disclosure to provide a method of processing Doppler OCT datasets in a manner that improves the signal to noise ratio of the resultant Doppler OCT images.

This Summary is provided to introduce a selection of concepts in a simplified form that are further described below in the Detailed Description. This Summary is not intended to identify key features or essential features of the disclosed subject matter, nor is it intended to be used to limit the scope of the disclosed subject matter. Furthermore, the disclosed subject matter is not limited to implementations that solve any or all disadvantages noted in any part of this disclosure.

### BRIEF DESCRIPTION OF THE SEVERAL VIEWS OF THE DRAWINGS

FIG. 1 is an illustration of en face Doppler technique for blood flow calculation in a vessel.

FIG. 2 is a scan pattern used for Doppler OCT. The raster scan, or 3D volume scan, covered a 2×2 mm area around the central retinal vessels and was repeated 5 times in 3 seconds.

FIG. 3A is an image of a flow phantom and OCT scanner.

FIG. 3B is an image of an articulating base.

FIG. 3C is a centration of OCT scan on tube of flow phantom (Left: SLO projection, Right: a horizontal OCT scan at the center of 3D scan pattern)

FIG. 3D is a diagram of the parabolic distribution of blood flow in the tube. The velocity at the center of tube is about 2 times of overall average speed V_{avg}.

FIG. 3E is a set of four images showing the transverse position of 3 frames (or B-scans) and their corresponding cross section images.

FIG. 4 is a plot of the pulsatility profile of artery and vein.

FIG. 5A is a flow chart used in detection of vessels.

FIG. 5B is a Doppler phase shift and reflectance Image on one en face plane.

FIG. 5C is a binary mask including the retina.

FIG. 5D is a vessel mask in one en face plane after vessel detection.

FIG. 5E is a Doppler OCT image of the vessel mask in FIG. 5D after phase unwrapping.

FIG. 5F is an illustration of two types of vein classification rules: vein (class 1) is the vein inside the disc, vein (class 2) is the vein outside the disc. At depth showed in this figure, the en face plane crosses the vein twice.

FIG. 5G is an image of two types of vein in the same en face plane.

FIG. 5H is an image of veins (class 1) detected in one en face plane (blue circle).

FIG. 6A is a set of three en face planes selected for comparison. Each vein had an optimized en face plane in which the Doppler phase shift was strong enough but without any artifacts. Blue circle is the valid vein mask. Red circle means the particular vessel obtained higher flow measurement than other planes.

FIG. 6B is an image of two branches merging to form one vessel in a deeper en face plane. The summation of branches (indicated by the upper two circles in FIG. 6A) are compared to the flow in the merged vessel (indicated by blue circle in FIG. 6B)

FIG. 7A is a Doppler OCT image using full spectrum phase resolve method (cross section), signal noise ratio (SNR) of Doppler phase shift=3.9.

FIG. 7B is a Doppler OCT image using split spectrum phase resolve method (cross section), SNR=7.9.

FIG. 8A is a plot of the relation between Doppler phase shift and speed. Fitting of estimated axial speed and averaged Doppler phase shift in the center of the tube. The blue line is the linear polynomial fitting (root measure square error RMSE=0.19) and the red line is the quadratic polynomial fitting with RMSE=0.17.

FIG. 8B is a plot showing the transformation between Doppler phase shift and speed.

FIG. 9 is a schematic of a system for processing OCT angiography data in accordance with the disclosure.

FIG. 10 is an example of a computing system in accordance with the disclosure.

FIG. 11 is flowchart showing a method for processing a volumetric OCT dataset to calculate TRBF.

### DETAILED DESCRIPTION

In the following detailed description, reference is made to the accompanying drawings which form a part hereof, and in which are shown by way of illustration embodiments that can be practiced. It is to be understood that other embodiments can be utilized and structural or logical changes can be made without departing from the scope. Therefore, the following detailed description is not to be taken in a limiting sense, and the scope of embodiments is defined by the appended claims and their equivalents.

Various operations can be described as multiple discrete operations in turn, in a manner that can be helpful in understanding embodiments; however, the order of description should not be construed to imply that these operations are order dependent.

The description may use the terms “embodiment” or “embodiments,” which may each refer to one or more of the same or different embodiments. Furthermore, the terms “comprising,” “including,” “having,” and the like, as used with respect to embodiments, are synonymous.

In various embodiments, structure and/or flow information of a sample can be obtained using OCT (structure) and Doppler OCT (flow) imaging-based on the detection of spectral interference. Such imaging can be two-dimensional (2-D) or three-dimensional (3-D), depending on the application. Structural imaging can be of an extended depth range relative to prior art methods, and flow imaging can be performed in real time. One or both of structural imaging and flow imaging as disclosed herein can be enlisted for producing 2-D or 3-D images. Unless otherwise noted or explained, all technical and scientific terms used herein are used according to conventional usage and have the same meaning as commonly understood by one of ordinary skill in the art which the disclosure belongs. Although methods, systems, and apparatuses/materials similar or equivalent to those described herein can be used in the practice or testing of the present disclosure, suitable methods, systems, and apparatuses/materials are described below.

All publications, patent applications, patents, and other references mentioned herein are incorporated by reference in their entirety. In case of conflict, the present specification, including explanation of terms, will control. In addition, the methods, systems, apparatuses, materials, and examples are illustrative only and not intended to be limiting.

In order to facilitate review of the various embodiments of the disclosure, the following explanation of specific terms is provided:

A-scan: A reflectivity profile that contains information about spatial dimensions and location of structures within an item of interest. An A-scan is an axial scan directed along the optical axis of the OCT device and penetrates the sample being imaged. The A-scan encodes reflectivity information (for example, complex numbers carrying intensity and phase information) as a function of depth.

B-scan: A cross-sectional tomograph that can be achieved by laterally combining a series of axial depth scans (i.e., A-scans) in the x-direction or y-direction. A B-scan encodes planar cross-sectional information from the sample and is typically presented as an image.

En face image: The planar set of voxels at a given depth along the z-axis of a volumetric 3D dataset (see below). In the present disclosure, the term “en face image” and “en face plane” are used to denote such data derived from 3D datasets of Doppler phase shift and/or structural OCT values.

Dataset: As used herein, a dataset is an ordered-array representation of stored data values that encodes relative spatial location in row-column-depth (x-y-z axes) format. In the context of OCT, as used herein, a dataset can be conceptualized as a three dimensional array of voxels, each voxel having an associated value (for example, a complex number, a phase shift, etc.). An A-scan corresponds to a set of collinear voxels along the depth (z-axis) direction of the dataset; a B-scan is made up of set of adjacent A-scans combined in the row or column (x- or y-axis) directions. Such a B-scan can also be referred to as an image, and its constituent voxels referred to as pixels. A collection of adjacent B-scans can be combined form a 3D volumetric set of voxel data referred to as a 3D image.

In the system and methods described herein, the dataset obtained by an OCT scanning device can be termed a “structural OCT” dataset whose values can, for example, be complex numbers carrying intensity and phase information. This structural OCT dataset can be used to calculate a corresponding dataset termed a “Doppler OCT” dataset comprised of Doppler phase shift values that are proportional to the axial component of flow within the imaged sample. There is a one-to-one correspondence between the voxels of the structural OCT dataset and the Doppler OCT dataset. Thus, segmented masks from a structural OCT image may be overlaid, for instance, on a corresponding Doppler OCT image to aid in image analysis.

An automated algorithm for the calculation of total retinal blood flow (TRBF) using optical coherence tomography (OCT) is disclosed. In an embodiment described herein, a commercial grade 70 kHz spectral domain OCT system was used to perform the TRBF quantification method. An exemplary method of using a flow phantom to calibrate the transformation from Doppler shift values to speed values is also described. In an embodiment, the OCT scan pattern can contain a plurality of repeated volume scans to improve calculation of TRBF. For example, a (2×2 mm) scan pattern centered on central retinal vessels in the optic disc can be used to a acquire data in 3 seconds for TRBF quantification. Such a scan pattern satisfies the balance required to collect data of sufficient quality with adequate coverage of vessels to perform the TRBF calculation method described herein while minimizing the scan time to which the patient must submit. In the disclosed methods, TRBF can be calculated using an en face Doppler technique. For each retinal vein detected in the acquired OCT scan, blood flow was measured at an optimal en face plane where the calculated flow was maximized. The TRBF was calculated by summing flow in all such veins. In an embodiment described herein, the algorithm tracked vascular branching so that either root or branch veins are summed, but never both. The TRBF in a plurality of repeated volumes can be averaged to reduce variation due to cardiac cycle pulsation. Finally, in embodiments, the TRBF can be corrected for eye length variation.

The disclosed algorithm has utility in detecting retinal blood flow differences for subjects with vascular pathology of the eye. In an exemplary demonstration described in greater detail below, twelve healthy eyes and 12 glaucomatous eyes were analyzed to test an embodiment of the disclosed algorithm. The TRBF was 45.4±6.7 μl/min for healthy control and 34.7±7.6 μl/min for glaucomatous participants (p-value=0.01). The intra-visit repeatability was 8.6% for healthy controls and 8.4% for glaucoma participants. Thus, the proposed automated method is able to provide repeatable TRBF measurement.

### Theory of En Face Method for TRBF Measurement

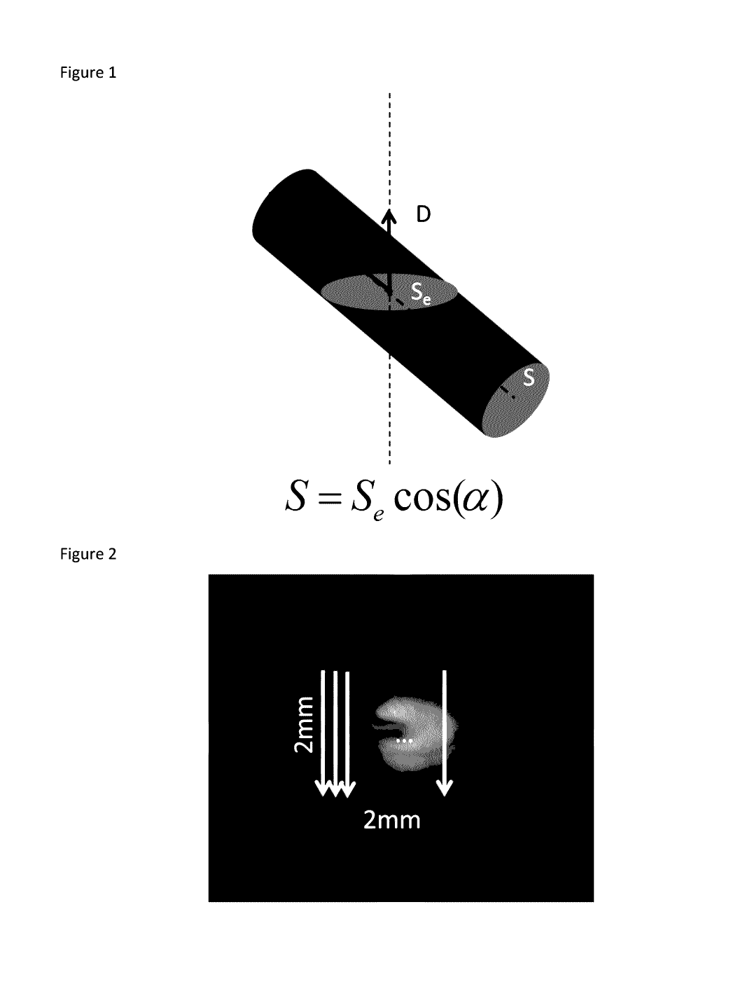

Flow calculation using Doppler OCT and the en face plane has been previously described (34, 36). Briefly (FIG. 1), the Doppler phase shift is proportional to the axial component of velocity, or DKV*cos(α), where D is Doppler phase shift, V is velocity, and a is the angle between axial component and vessel. The true vessel area, which is perpendicular to the velocity vector in vessels, is equal to the product of vessel area in the en face plane and cos(α), or S=Sₑ*cos(α). Here, S is the area of a vessel in the direction perpendicular to the vessel segment, and Sₑ is the area of the vessel in the en face plane. When the integration of V in S is performed, the two cos(α) components cancel each other. Thus, blood flow in a vessel is proportional to the integration of Doppler phase shift in the vessel region and the en face plane.

### Doppler OCT and Scan Pattern

The OCT system used in the exemplary study described herein was an RTVue-XR spectral domain OCT, a commercially-available device for ophthalmic imaging applications. The RTVue-XR has a 70 kHz scan rate, a laser source with central wavelength of 840 nm and 5 micron depth resolution. The phase wrapping velocity limit of RTVue-XR is 11.1 mm/s using the formula Vmax=λ/(4nΔT) and wavelength λ=840 nm, refraction index n=1.36 and axial scan (A-scan) time interval ΔT=14.3 μs. The scan pattern using this system for TRBF measurement included 5 volume scans covering a 2×2 mm area around the optic disc. The volume scan contained 80 B-scans and each B-scan contained 500 axial-scans. The scan depth was 2.3 mm. The volume scan was repeated 5 times in the scan pattern and took 3 seconds to obtain (FIG. 2).

The blood velocity in the central retinal veins has been reported to be as high as 57 mm/s based on ultrasound measurements. Because the central retinal vein is oriented along the axial direction, axial speeds of this magnitude would exceed multiple phase wrapping limit of a 70-kHz OCT system. The average speed in peripapillary veins is 15 to 16.8 mm/s as assessed by Doppler OCT. In this region, the vessels are oriented more closely to perpendicular to the axial direction. Thus, the axial speed in peripapillary veins may be too low to allow for precise speed measurement. Therefore, the vein branches near the disc boundary, where blood speeds fall between those of the peripapillary veins and central retinal veins, were targeted. The axial speed in these branches near the disc boundary can be reliably measured when the angle is appropriate in some en face planes.

### Doppler Phase Shift Calculation and Bulk Motion Removal

In an embodiment disclosed herein, the Doppler phase shift was calculated based on the phase-resolved technique (18, 23). With the use of faster scan rates, however, the noise can be increased compared to earlier slower OCT systems (38). In an embodiment disclosed herein, in order to reduce the noise in the OCT image, a split spectrum method adapted for use in Doppler OCT was applied. A filter bank was applied to divide the full-spectrum interferograms into a number of narrower bands (39,40). In embodiments, the filter bank can consist of a plurality of bandpass filters with variable degrees of overlap along the frequency spectrum. The use of such a split spectrum method reduces noise by averaging the Doppler phase shift in all bands. This noise reduction also produces the tradeoff of lower depth resolution due to the small spectral bandwidth in each band.

The split spectrum was generated by passing a full-spectrum fringe through the filter bank. Then the FFT transform was applied to each band separately (Equation 1).

s(x,j,z)=FFT(S(x,k) [symbol] f(j,k)) (1)

Here, S(x,k) is the spectrum obtained for xₜₕ A-scan in k-space, f(j,k) is the jₜₕ band pass filter, FFT is the fast Fourier transform, s(x,j,z) is the complex signal of jₜₕ band of xₜₕ A-scan.

Doppler phase shift was then averaged in all bands and neighboring A-scans (Equation 2).

$$D ( x , z ) = \frac{arg ( {\sum }_{i = 0}^{N - 1} {\sum }_{j = 1}^{M} s ( x + i , j , z ) {s}^{*} ( x + i + 1 , j , z ) )}{2 Δ T} \qquad ( 2 )$$

Here, D (x,z) is the Doppler phase shift of xₜₕ A-scan at depth position z, arg is a function to give the angle of a complex number, N is the number of neighboring A-scans, M is the number of bands, ΔT is the time difference between xₜₕ A-scan and (x+1)ₜₕ A-scan. By averaging the N A-scans and M bands, the noise of the Doppler OCT image was reduced.

In embodiments, bulk motion due to eye movement along the depth direction can be calculated and removed using the averaged Doppler phase shift of the static retinal tissue as the bulk motion induced phase shift. In an embodiment described herein, a revised histogram estimation method for bulk motion was used (41). The original method of (41) calculated the histogram of Doppler phase shift in static retinal tissue for an A-scan, and used the value of the maximum bin as bulk motion. In regions with less effective data points such as regions with large vessels and a thin retina, the method may be not precise. As each B-scan was acquired in less than 10 milliseconds and the bulk motion changed gradually along the B-scan, a weighted polynomial fitting was applied on the bulk motion estimated by the original method. To reduce the error, weights were set to 0 for A-scans with less effective points.

### Doppler Phase Shift Calibration

Beyond the phase wrapping limit, fringe washout reduces OCT signal strength and increases phase noise. Therefore phase-unwrapping algorithms often do not recover the full Doppler shift signal and have a tendency to underestimate flow speed. It has been reported that the relationship between Doppler shift obtained from the phase-unwrapping method and the actual speed was not perfectly linear (42). Further, when the space interval between two consecutive axial scans increases, the correlation between two axial scans becomes weak and noise of the estimated Doppler phase shift increases. Therefore the summation of Doppler phase shift in the vessel area will be smaller than actual Doppler phase shift produced by blood flow. This phenomenon has been called step-size factor and needs to be compensated for (23). In an exemplary embodiment described herein, compensation for both biases can be achieved by empirical calibration using a physical flow phantom.

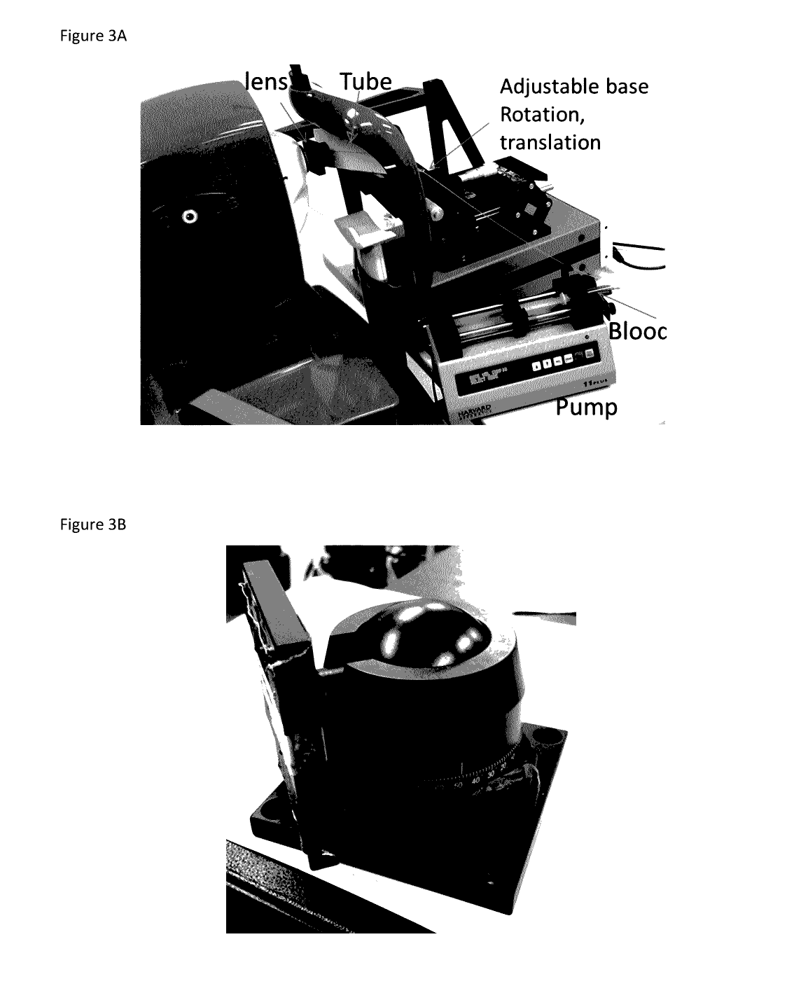

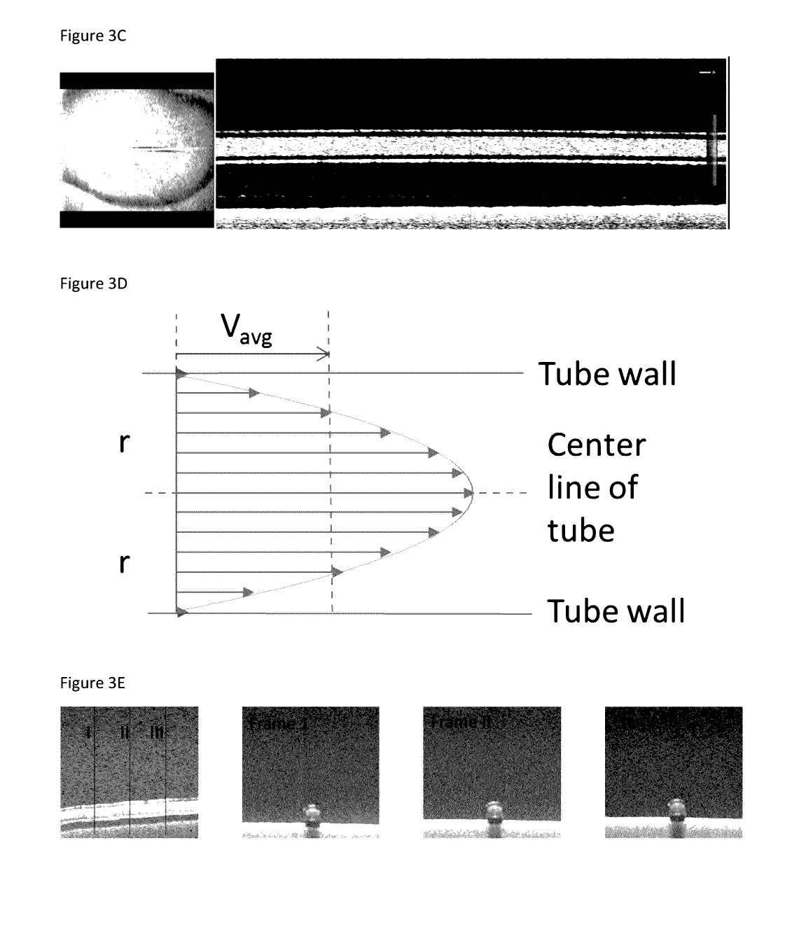

An exemplary procedure for determining such an empirical calibration is as follows. A syringe was filled with fresh Bovine blood (Animal Technologies, Inc., Tyler, Tex.) and then pumped by a syringe pump (11 plus, Harvard Apparatus, Holliston, Mass.). The blood was pumped with a constant speed through a plastic tube with a 200 μm inner diameter (FIG. 3A). The tube was fixed in front of the OCT scanner. The angle between the tube and laser beam could be adjusted using an articulating base (SL20, Thorlabs Inc, Newton, N.J.) (FIG. 3B). An achromatic doublet (AC254-030-B, Thorlabs Inc, Newton, N.J.) was placed between the OCT scanner and tube in order to focus the laser beam on to the tube. The same scan pattern described earlier for TRBF measurement was used to obtain the 3D image of the tube. In the experiment, the pump flow rate was set to be 60 μl/min, which corresponds to an average velocity of 31.8 mm/s in the 200 μm tube. The speed was specifically chosen to simulate the blood flow rate in retina vessels inside optic disc. The vertical and horizontal position of the tube was adjusted to center it with laser beam (FIG. 3C). Then the incident angle was changed from 0 to 18 degrees using steps of 3 degrees. The maximum angle was limited to 18 degrees because the reflectance signal is too weak due to fringe washout when angle is larger than 18 degrees.

The same TRBF scan pattern used in the herein described human study was also used for the phantom test in order to maintain a common A-scan space interval. Therefore compensation for the step-size factor after the calibration need not be considered. The Doppler phase shift was calculated as described above. Due the difference in the reflectance index between air, tube, and blood, there is significant shape distortion at the edges of the tube where the incident angle is much larger than zero. To avoid this distortion, only signal in the center of the tube was used where the incident angle is close to 0 and the distortion is negligible. The flow in the tube followed a parabolic distribution (FIG. 3D) along the tube radial direction, in accordance with fluid mechanics theory. Therefore the Doppler shift in the center of tube was larger than at the edges. The theoretical average speed in the central region with radius <20 micron was recalculated to be 61.4 mm/s. The average velocity was obtained by averaging the Doppler shift in the center part of tube in the Doppler OCT image. Image segmentation was applied on reflectance images to find the top position of tube on each B-scan. Then the beam incident angle was estimated based on the 3D orientation of the tube. (FIG. 3E). The phase unwrapping algorithm is used when it is necessary. The axial speed was then calculated based on the Doppler shift and estimated incident angle.

### Pulsatility in Vessels

Pulsatility during the cardiac cycle makes the blood flow in the retina variable. To obtain repeatable TRBF quantification, multiple volumetric scans are needed to match the different time points in the cardiac cycle. In an embodiment using the OCT system described previously for the studies disclosed herein, in order to cover the entire optic disc, the scan size needed to be about 2×2 mm. The A-scan interval was limited to ensure the correlation of neighboring A-scans. The B-scan interval was also limited to ensure middle size vessels were included (D>50 micron). Therefore, a scan pattern with 80 B-scan*500 A-scans, covering 2×2 mm area was chosen. The volumetric scan rate was 1.7 Hz.

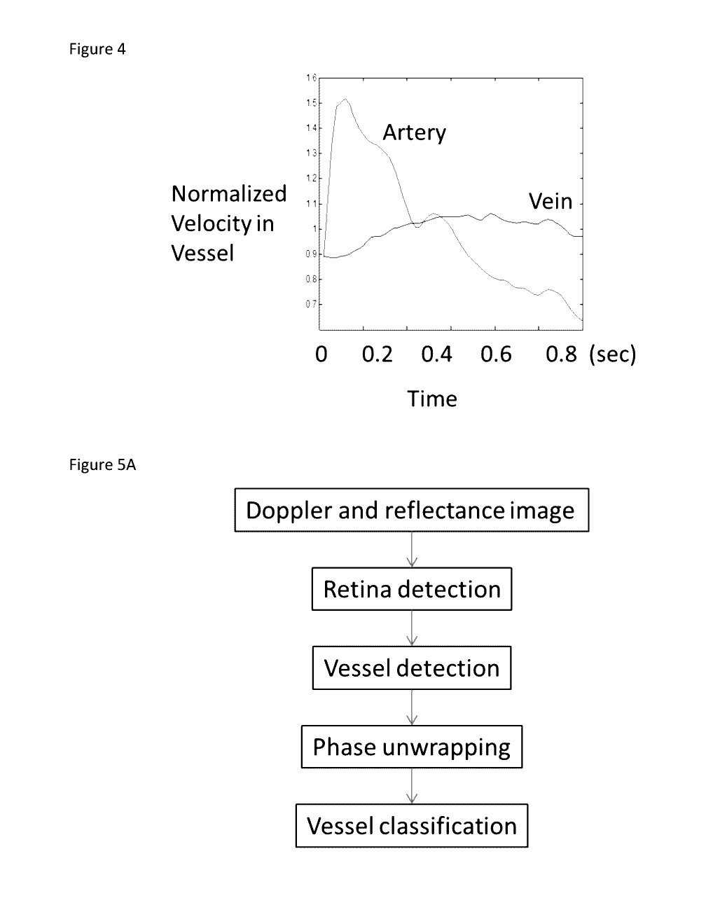

The pulsatility of an artery cannot be compensated with such a volumetric scan rate. The pulsatility profile of a retinal artery and vein were compared in an exemplary experiment using a rapidly repeating circular scan. Eyes were scanned by repeating circular scans with a 1.5 mm diameter at 30 Hz. In each cross-sectional image, the Doppler phase shift in the artery and vein was summated in the vessel area and normalized by its own mean. The start position of each cardiac cycle was detected using the maximum change in artery flow profile. Then, the profile of all cardiac cycles were aligned and averaged. By this method, an average pulsatility profile of an artery and a vein in one cardiac cycle was determined (FIG. 4). The variance in an artery was quite large compared to its mean (coefficient of variation=0.26), while the variance in a vein was relatively small compared to its mean (coefficient of variation=0.05). Thus, the pulsatility variation can be compensated for if venous flow is measured and a sufficient number of volumes acquired. The slower flow speed in veins also caused less phase wrapping than in arteries.

### Vessel Detection and Phase Unwrapping

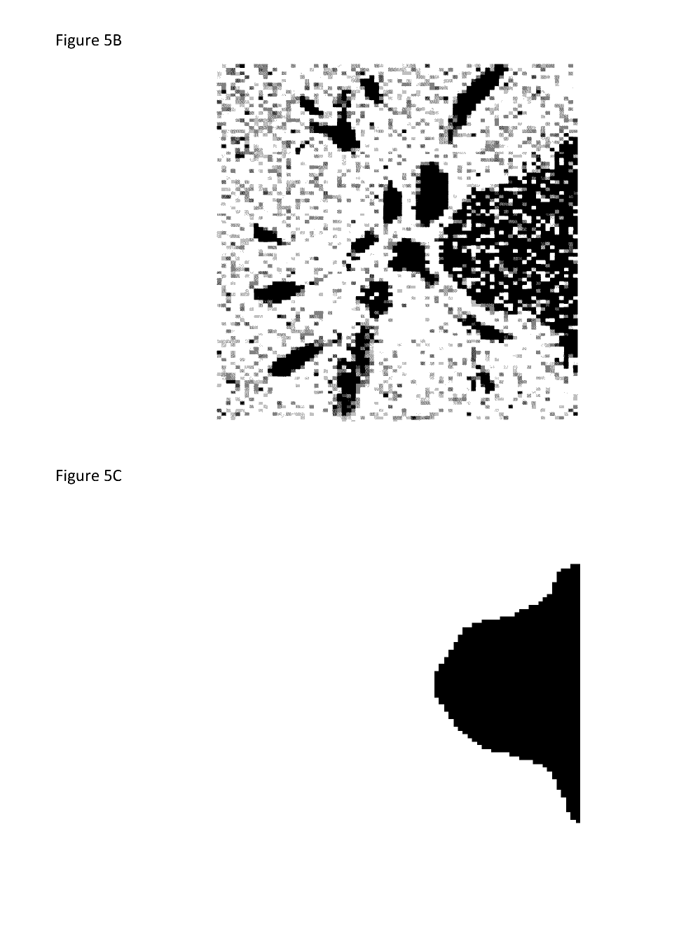

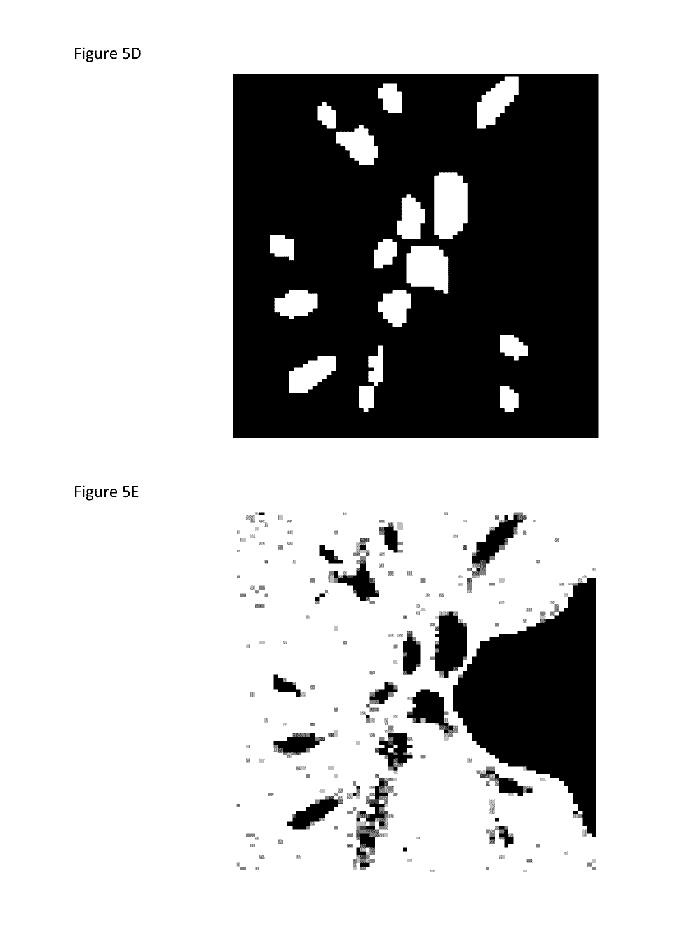

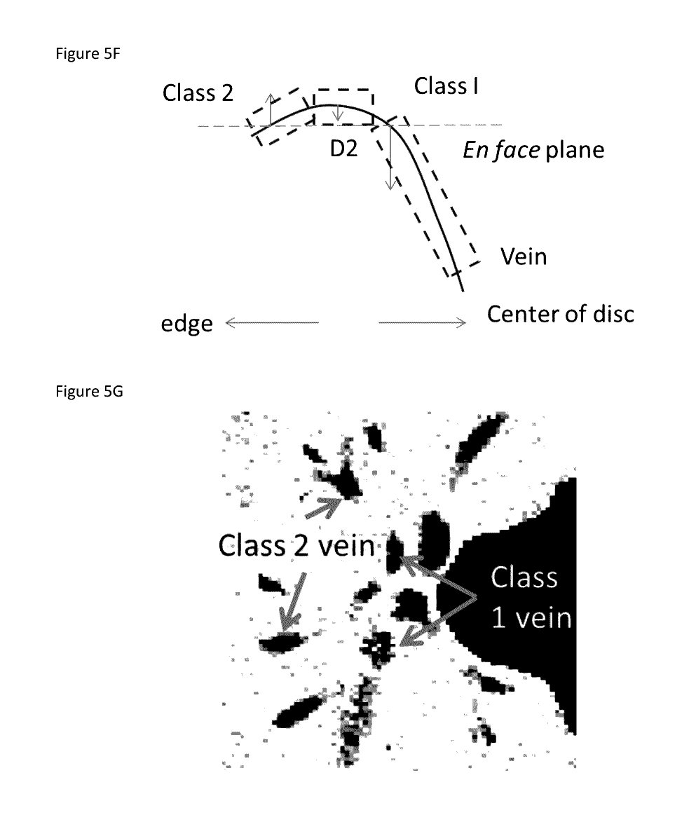

In order to detect veins, an automated algorithm was developed (FIG. 5A). In an embodiment of such a detection algorithm, the retina can first be detected using the reflectance images from the structural OCT scan (FIG. 5C). This detection can be performed using appropriate image segmentation algorithms known in the art. In the example disclosed herein, a level-set method was applied to obtain a smooth binary retinal mask (43). The level-set method sometimes created multiple regions that were not connected, for instance, when there was extra tissue above the retina. In such cases, the region with maximum volume was classified as the retina mask.

In an embodiment, vessels are detected inside the retina mask as voxels having large Doppler phase shifts (i.e., having Doppler shift values exceeding a prescribed detection threshold). In the example embodiment described herein, the detection threshold was defined as 2.33 times the standard deviation of Doppler phase shift in the static retinal tissue. In further embodiments, morphologic operations can then be used to exclude small regions, close holes, and/or connect nearby regions (for example, erosion and dilation operators can be applied) to produce a vessel mask. In the specific embodiment described herein, where the spatial interval between two adjacent B-scans was 25 microns, it was difficult to decide if a region with 1-2 pixels was noise or signal. Therefore any region with a diameter <=50 micron was removed from the vessel mask (FIG. 5D).

Phase wrapping may change the sign of Doppler phase shift in the center of the vessel. Thus, in an embodiment, an unsupervised phase unwrapping algorithm (44, 45) is applied before the vein and artery classification. In the implementation described herein, the phase unwrapping algorithm was applied to the 2D image in each en face plane separately, and was only applied to pixels inside the retina mask (FIG. 5E).

In an embodiment, after the vessels are detected and the phase unwrapping algorithm applied, classification of vessels as veins and arteries is performed. In an implementation described herein, the following classification scheme is used: from the vessel mask, arteries and veins which were double counted in the same en face plane were excluded. Veins were identified as vessels having flow directed into the disc. The determination of vessel type is illustrated in FIG. 5F. As shown, a typical vein has three segments D1-D3 and the corresponding Doppler phase shifts in these segments were negative, close to 0, and positive.

The algorithm in the previous paragraph detected two vessel segments: the first one corresponds to D1, or class 1, where both the Doppler phase shift and the slope of the vessel segment are negative; the other one corresponds to D3, or class 2, where both the Doppler phase shift and slope of vessel segment are positive (FIG. 5G). Vessel segment D2 cannot be detected because the Doppler phase shift is too weak. In arteries, however, vessel segment D1 has a positive Doppler phase shift and a negative slope of vessel segment, while vessel segment D3 has a negative Doppler phase shift and a positive slope of vessel segment. In an embodiment described herein, only vessel segments with both negative Doppler phase shift and negative slope of vessel segment are considered as valid venous mask for TRBF measurement (FIG. 5H).

### Multiple Planes En Face Doppler Method

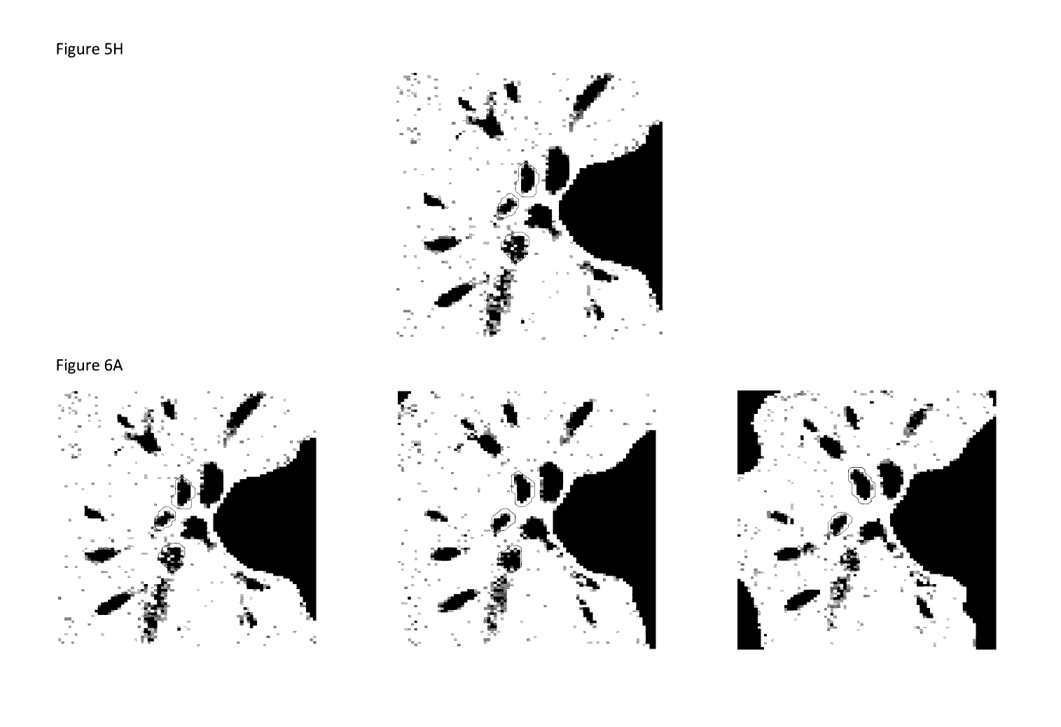

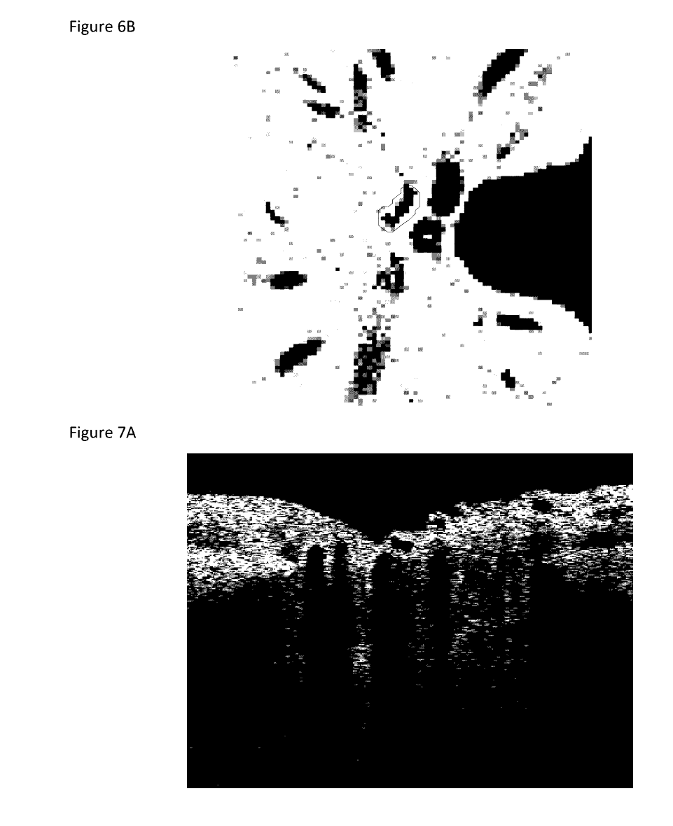

Due to artifacts in the spectral OCT (such as fringe washout, phase wrapping or weaker reflectance in deeper tissue) it is difficult to find a single artifact-free plane that includes all veins classified for inclusion in TRBF measurement. To solve this problem, in an embodiment, an optimized en face plane for each separate vein branch can be determined and then the flow contribution of all separate vein branches summed (FIG. 6A).

In an embodiment, the optimized en face plane for a given vein branch is the plane at the depth in the tissue for which the blood flow is maximum. On other, non-optimum, en face planes, artifacts reduce the integration of the Doppler phase shift over the vessel area and thus create lower blood flow measurement than in the optimized en face plane (FIG. 6A). Total retinal blood flow can then be calculated by summing the optimized flow value of each vein branch. In an embodiment, if two vein branches are found to merge into a root vein at a deeper level, the summation of flow values in the branches is compared to the flow value quantified in the root vein. Either the root or branch veins are then selected for inclusion in the calculation of total retinal blood flow, depending on which one is larger (FIG. 6B). In an embodiment, to reduce the effect of cardinal pulsation, TRBF values calculated from a plurality of volumes acquired during one scan session can be averaged to obtain a more reliable TRBF measurement.

The pixel size of the en face plane is proportional to the axial length of the eye. Since axial length is variable, a compensation factor is necessary to correct TRBF using a default pixel size. In an embodiment, a compensation factor c=(L/L_{d})², where L is the axial length of the test eye and L_{d} is the default axial length is used. For the exemplary implementation described in the studies herein, a value of L_{d}=24 mm was used. This compensation method for vessel area was consistent to methods used in other imaging modalities (46).

### EXAMPLES

The following examples are illustrative of the disclosed methods. In light of this disclosure, those of skill in the art will recognize that variations of these examples and other examples of the disclosed method would be possible without undue experimentation.

### Example 1

### Improved Signal-to-Noise Ratio Using Split-Spectrum Doppler Phase Shift

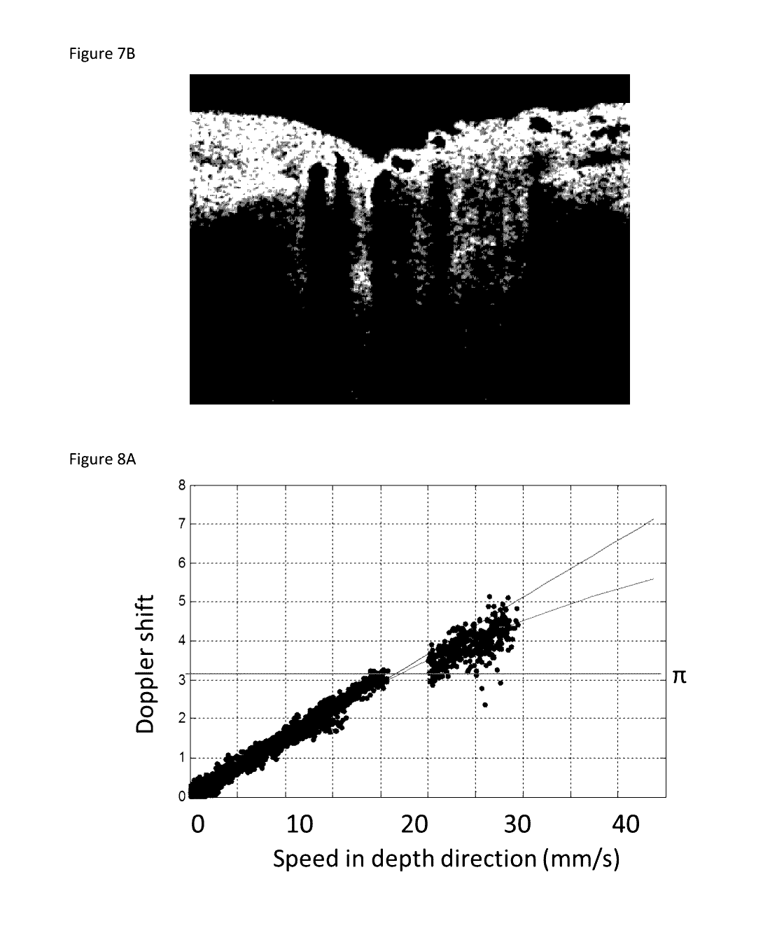

In the split spectrum algorithm for Doppler phase shift calculation (Equation 2 above), the number of A-scans to be averaged was set at N=6 and the number of bands to be averaged was set at M=11. For comparison, the signal to noise ratio (SNR) was calculated for both the traditional full spectrum algorithm and the split spectrum algorithm disclosed herein. The SNR is the ratio between the average of Doppler phase shift in a vessel and the standard deviation of Doppler phase shift in the non-vessel static retinal tissue, where the Doppler phase shift was supposed to be zero due to lack of motion. The SNR of Doppler phase shift for split spectrum algorithm was larger than traditional phase-resolved algorithm (7.9 vs 3.9) (FIG. 7A and FIG. 7B). The improvement of SNR was, however, at the cost of resolution in depth. The depth resolution using the split spectrum is 20 microns compared to the original 5 micron depth resolution. The reduction of depth resolution did not affect the blood flow calculations because blood flow was calculated in the en face plane using adjacent A-scans. However, loss of depth resolution can potentially limit the selection of the proper en face planes for vein branch calculations of blood flow.

### Example 2

### Calibration Experiment for Transformation of Doppler Shift to Speed

The phantom experiments showed a linear relationship between the Doppler phase shift and the flow speed when the absolute value of the phase shift was less than n (FIG. 8A). In FIG. 8A, each data point corresponds to the average of Doppler phase shift in a cross section plane of the center of the tube. The axial speed was calculated by 61.4 mm/s*sin(β), where (3 was estimated by the slope of a fitted line of detected center of tube in the 3D volume. The speed corresponding to Doppler phase shift n was 18.5 mm/s, much larger than theoretic value of 11.1 mm/s. This difference was possibly due to the larger A-scan space interval in the scan pattern (about 4 micron). When the Doppler phase shift was larger than n, the distribution of data points became more scattered. A nonlinear fitting (red line) worked better than a linear fitting (blue line). In the studies described herein, a nonlinear curve was used for transforming Doppler phase shift values to speed (FIG. 8B).

### Example 3

### TRBF Measurement in Healthy Versus Glaucomatous Eyes

To test the ability of the proposed TRBF measuring algorithm to detect differences in vascular function, participants were selected from functional and structural OCT for glaucoma study in Casey Eye Institute. The research protocol was approved by the institutional review boards at Oregon Health & Science University and carried out in accordance with the tenets of the Declaration of Helsinki. Written informed consent was obtained from each subject.

In the described studies, normal participants had IOPs of less than 21 mm Hg for both eyes, a normal Humphrey Swedish Interactive Threshold Algorithm (SITA) 24-2 standard visual field with mean deviation (MD), and pattern standard deviation (PSD) within 95% limits of the normal reference. They also had a glaucoma hemifield test within 97% limits, a central corneal thickness ≧500 μm, a normal appearing ONH, a normal nerve fiber layer, an open anterior chamber angle as observed by gonioscopy, and no history of chronic ocular or systemic corticosteroid use.

The glaucoma participants had at least one eye which had ONH or NFL defect visible on slit-lamp biomicroscopy defined as one of following: diffused or localized rim thinning, disc (splinter) hemorrhage, vertical cup/disc ratio greater than the fellow eye by >0.2, or a notch in the rim detected on baseline dilated fundus examination and confirmed by masked reading of stereo disc photographs. People were not enrolled if they had other eye diseases, previous intraocular surgery, age <40 or >79 years, or best corrected visual acuity worse than 20/40.

The TRBF measurement algorithm was applied to 12 healthy and 12 glaucomatous participants. One eye was selected for each participant. The proposed scan pattern with 5 volumes was applied to the tested eye. Three scans were repeated for each eye. The disclosed multiple plane en face algorithm was applied for TRBF calculation. The average age was 64±7 for healthy controls and 70±9 for glaucomatous participants. The visual field mean deviation was 0.7±1.4 dB for healthy controls and −6.1±7.1 dB for glaucomatous participants. The TRBF was 45.4±6.7 μl/min for healthy control and 34.7±7.6 μl/min for glaucomatous participants (p-value=0.01). The intra-visit repeatability (coefficient of variation) is 8.6% for healthy controls and 8.4% for glaucomatous participants.

### Example 4

### Optical Coherence Tomography Angiography Image Processing System

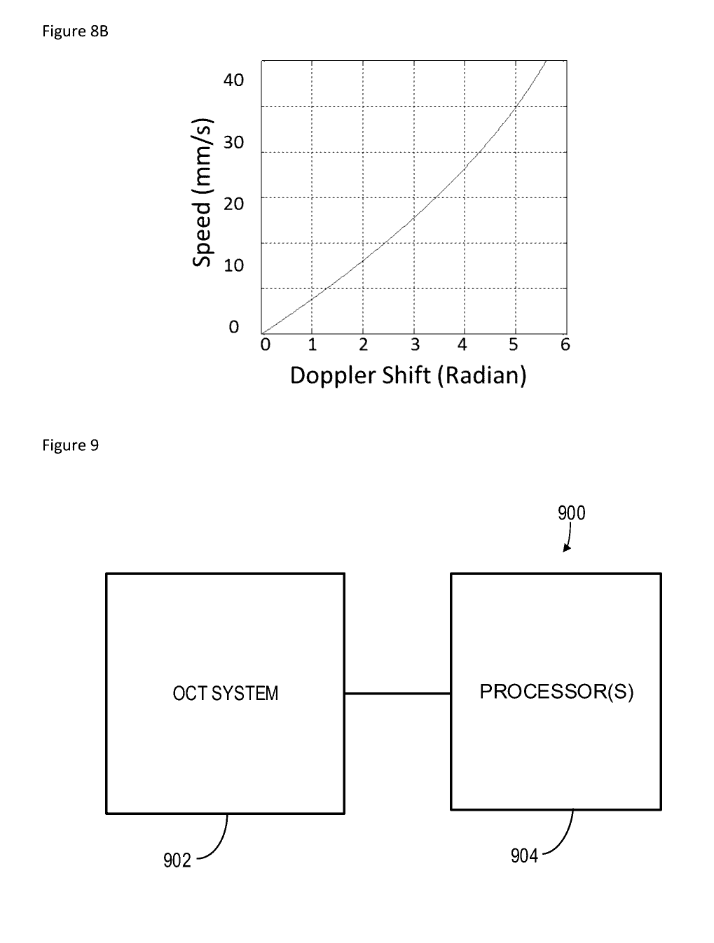

FIG. 9 schematically shows an example system 900 for OCT image processing in accordance with various embodiments. System 900 comprises an OCT system 902 configured to acquire an OCT image comprising OCT interferograms and one or more processors or computing systems 904 that are configured to implement the various processing routines described herein. OCT system 900 can comprise an OCT system suitable for OCT angiography applications, e.g., a swept source OCT system.

In various embodiments, an OCT system can be adapted to allow an operator to perform various tasks. For example, an OCT system can be adapted to allow an operator to configure and/or launch various ones of the herein described methods. In some embodiments, an OCT system can be adapted to generate, or cause to be generated, reports of various information including, for example, reports of the results of scans run on a sample.

In embodiments of OCT systems comprising a display device, data and/or other information can be displayed for an operator. In embodiments, a display device can be adapted to receive an input (e.g., by a touch screen, actuation of an icon, manipulation of an input device such as a joystick or knob, etc.) and the input can, in some cases, be communicated (actively and/or passively) to one or more processors. In various embodiments, data and/or information can be displayed, and an operator can input information in response thereto.

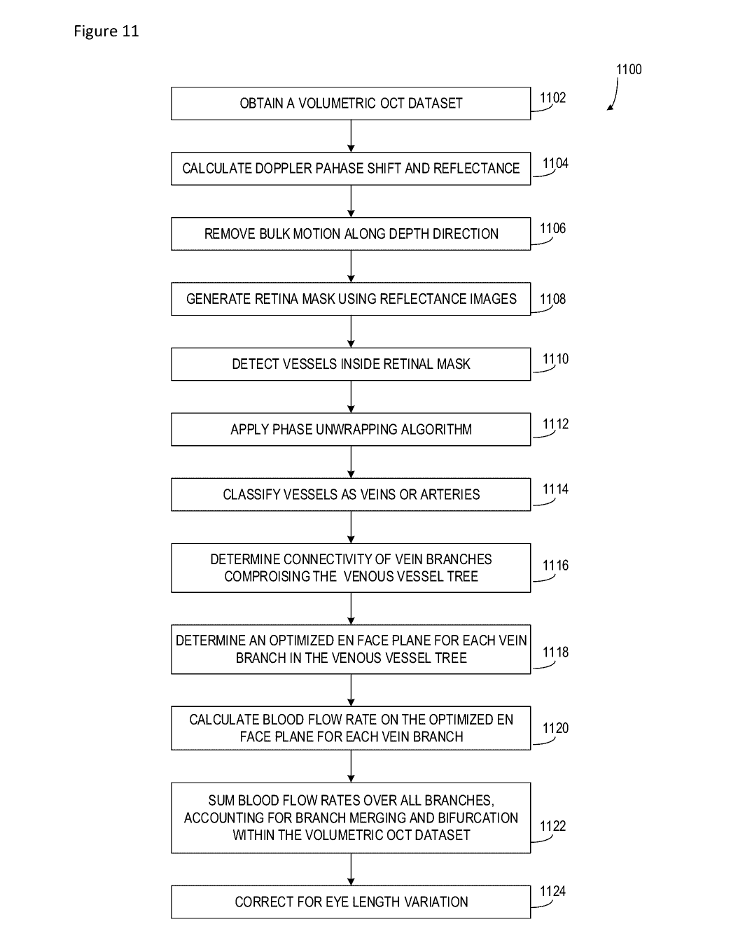

In some embodiments, the above described methods and processes can be tied to a computing system, including one or more computers. In particular, the methods and processes described herein, e.g., the methods depicted in FIG. 5A described above or in FIG. 11 described below, can be implemented as a computer application, computer service, computer API, computer library, and/or other computer program product.

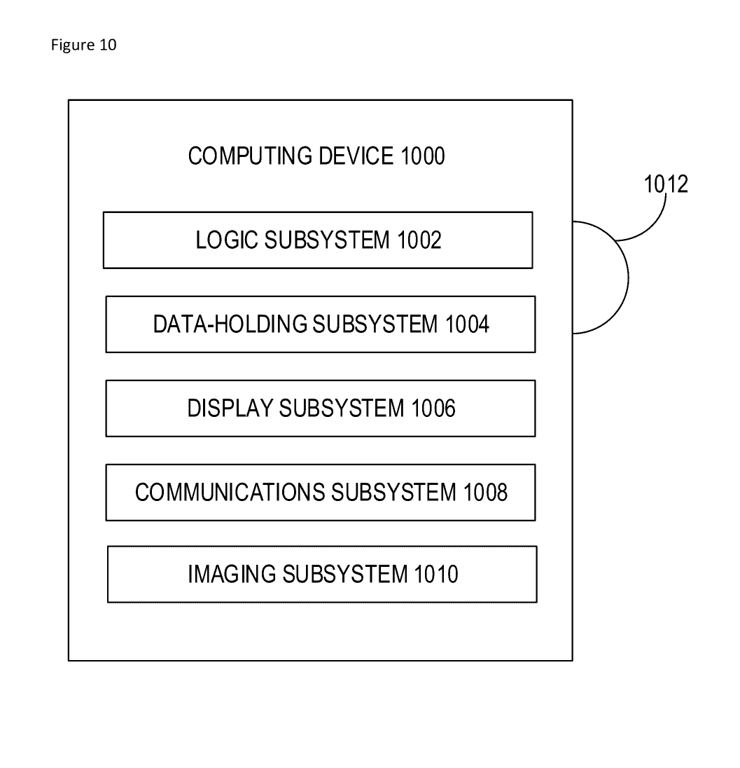

FIG. 10 schematically shows a non-limiting computing device 1000 that can perform one or more of the above described methods and processes. For example, computing device 1000 can represent a processor included in system 900 described above, and can be operatively coupled to, in communication with, or included in an OCT system or OCT image acquisition apparatus. Computing device 1000 is shown in simplified form. It is to be understood that virtually any computer architecture can be used without departing from the scope of this disclosure. In different embodiments, computing device 1000 can take the form of a microcomputer, an integrated computer circuit, printed circuit board (PCB), microchip, a mainframe computer, server computer, desktop computer, laptop computer, tablet computer, home entertainment computer, network computing device, mobile computing device, mobile communication device, gaming device, etc.

Computing device 1000 includes a logic subsystem 1002 and a data-holding subsystem 1004. Computing device 1000 can optionally include a display subsystem 1006, a communication subsystem 1008, an imaging subsystem 1010, and/or other components not shown in FIG. 7. Computing device 1000 can also optionally include user input devices such as manually actuated buttons, switches, keyboards, mice, game controllers, cameras, microphones, and/or touch screens, for example.

Logic subsystem 1002 can include one or more physical devices configured to execute one or more machine-readable instructions. For example, the logic subsystem can be configured to execute one or more instructions that are part of one or more applications, services, programs, routines, libraries, objects, components, data structures, or other logical constructs. Such instructions can be implemented to perform a task, implement a data type, transform the state of one or more devices, or otherwise arrive at a desired result.

The logic subsystem can include one or more processors that are configured to execute software instructions. For example, the one or more processors can comprise physical circuitry programmed to perform various acts described herein. Additionally or alternatively, the logic subsystem can include one or more hardware or firmware logic machines configured to execute hardware or firmware instructions. Processors of the logic subsystem can be single core or multicore, and the programs executed thereon can be configured for parallel or distributed processing. The logic subsystem can optionally include individual components that are distributed throughout two or more devices, which can be remotely located and/or configured for coordinated processing. One or more aspects of the logic subsystem can be virtualized and executed by remotely accessible networked computing devices configured in a cloud computing configuration.

Data-holding subsystem 1004 can include one or more physical, non-transitory, devices configured to hold data and/or instructions executable by the logic subsystem to implement the herein described methods and processes. When such methods and processes are implemented, the state of data-holding subsystem 1004 can be transformed (e.g., to hold different data).

Data-holding subsystem 1004 can include removable media and/or built-in devices. Data-holding subsystem 1004 can include optical memory devices (e.g., CD, DVD, HD-DVD, Blu-Ray Disc, etc.), semiconductor memory devices (e.g., RAM, EPROM, EEPROM, etc.) and/or magnetic memory devices (e.g., hard disk drive, floppy disk drive, tape drive, MRAM, etc.), among others. Data-holding subsystem 1004 can include devices with one or more of the following characteristics: volatile, nonvolatile, dynamic, static, read/write, read-only, random access, sequential access, location addressable, file addressable, and content addressable. In some embodiments, logic subsystem 1002 and data-holding subsystem 1004 can be integrated into one or more common devices, such as an application specific integrated circuit or a system on a chip.

FIG. 10 also shows an aspect of the data-holding subsystem in the form of removable computer-readable storage media 1012, which can be used to store and/or transfer data and/or instructions executable to implement the herein described methods and processes.

Removable computer-readable storage media 1012 can take the form of CDs, DVDs, HD-DVDs, Blu-Ray Discs, EEPROMs, flash memory cards, USB storage devices, and/or floppy disks, among others.

When included, display subsystem 1006 can be used to present a visual representation of data held by data-holding subsystem 1004. As the herein described methods and processes change the data held by the data-holding subsystem, and thus transform the state of the data-holding subsystem, the state of display subsystem 1006 can likewise be transformed to visually represent changes in the underlying data. Display subsystem 1006 can include one or more display devices utilizing virtually any type of technology. Such display devices can be combined with logic subsystem 1002 and/or data-holding subsystem 1004 in a shared enclosure, or such display devices can be peripheral display devices.

When included, communication subsystem 1008 can be configured to communicatively couple computing device 1000 with one or more other computing devices. Communication subsystem 1008 can include wired and/or wireless communication devices compatible with one or more different communication protocols. As non-limiting examples, the communication subsystem can be configured for communication via a wireless telephone network, a wireless local area network, a wired local area network, a wireless wide area network, a wired wide area network, etc. In some embodiments, the communication subsystem can allow computing device 1000 to send and/or receive messages to and/or from other devices via a network such as the Internet.

When included, imaging subsystem 1010 can be used acquire and/or process any suitable image data from various sensors or imaging devices in communication with computing device 1000. For example, imaging subsystem 1010 can be configured to acquire OCT image data, e.g., interferograms, as part of an OCT system, e.g., OCT system 902 described above. Imaging subsystem 1010 can be combined with logic subsystem 1002 and/or data-holding subsystem 1004 in a shared enclosure, or such imaging subsystems can comprise periphery imaging devices. Data received from the imaging subsystem can be held by data-holding subsystem 1004 and/or removable computer-readable storage media 1012, for example.

### Example 5

### Processing Steps for TRBF Calculation

FIG. 11 shows an exemplary flowchart of a method 1100 for calculating TRBF from an acquired volumetric OCT dataset 1102 using the methods disclosed herein. In practice, this method 1100 can be applied to a plurality of volumetric OCT datasets acquired at the same location to produce a set of calculated TRBF values, which can further be averaged to reduce the variation caused by cardiac cycle pulsation effects. At 1104, datasets for the Doppler phase shift and reflectance images are calculated. In embodiments, the Doppler phase shift can be calculated using methods known in the art, or by application of Equations (1) and (2) to split the spectrum and reduce noise in the resultant Doppler phase shift data. At 1106, an algorithm for removal of bulk motion is applied. At 1108, reflectance images are used to detect the retina and generate a retina mask within which vessel detection will be performed. Generation of this retinal mask can be performed using any segmentation approach which provides suitable delineation of retinal versus non-retinal tissue. Examples of such segmentation approaches include levels-set methods, active contours, thresholding approaches, clustering methods, image growing, or other standard segmentation methods known in the art.

At 1110, the retinal mask generated at 1108 is applied to images to constrain the search space to detect vessels within the Doppler phase shift dataset. In an embodiment, voxels having large phase shift values (i.e., above a given threshold) can be classified as belonging to a blood vessel structure. In such an embodiment, the threshold value can be determined from the Doppler phase shift values associated with static (i.e., non-flowing) retinal tissue. For example, the standard deviation (SD) of the Doppler phase shift values of for static retinal tissue can be calculated, and then a threshold value set at a multiple of that SD value (e.g., about 2.3 times SD). Once identified, these detected vessel voxels are used to generate a set of en face vessel image masks for the dataset. In embodiments, it may be further necessary to perform morphological operations (e.g., dilation, erosion) to improve the quality of the en face vessel mask images by filling holes, restoring connectivity, and rejecting vessels below a certain size. At 1112 a phase unwrapping algorithm is applied to each of the en face images in the dataset, with the phase unwrapping performed only on pixels lying within the retinal mask for that en face image.

At 1114, the vessel masks are used with the en face Doppler phase shift images of the dataset to classify detected vessels as veins or as arteries. In an embodiment (as described earlier), this classification can be performed using knowledge the measured flow direction (i.e., whether the Doppler phase shift is positive or negative) and the spatial slope of a vessel relative to the location of the vessel trunk in the optic nerve head. At 1116, the subset of vessels classified as veins in the en face images are analyzed to characterize the spatial connectivity of the venous vessel tree through the depth of the dataset. This characterization establishes the specific branches comprising the venous vessel tree and locations where branches merge or bifurcate.

At 1118, each of the branches of the venous vessel tree is analyzed to determine an optimized en face plane depth at which the blood flow rate for that branch is calculated. In an embodiment, the optimized en face plane depth is defined as the en face depth along the vein branch where the calculated blood flow rate is maximum. Because of geometric orientation, artifacts, and noise, the optimized en face plane depth is expected to be different for each vein branch within the venous vessel tree. At 1120, the blood flow rate is calculated for each vein branch at its respective optimized en face plane by converting the Doppler phase shift values within that vein branch to speed (for example, by using an empirical relationship such as that depicted in FIG. 8b), and then integrating those speed values over the en face cross sectional area of the vein branch. Once all optimized flow rates are determined for all vein branches, and allowances made for cases where veins merge or bifurcate, a total retinal blood flow (TRBF) value is calculated at 1122 by summing the flow contributions of the individual branches. Lastly, at 1124, scaling is performed to account for variation in eye length between subjects. It is to be understood that the configurations and/or approaches described herein are exemplary in nature, and that these specific embodiments or examples are not to be considered in a limiting sense, because numerous variations are possible. The specific routines or methods described herein can represent one or more of any number of processing strategies. As such, various acts illustrated can be performed in the sequence illustrated, in other sequences, in parallel, or in some cases omitted. Likewise, the order of the above-described processes can be changed.

The subject matter of the present disclosure includes all novel and nonobvious combinations and subcombinations of the various processes, systems and configurations, and other features, functions, acts, and/or properties disclosed herein, as well as any and all equivalents thereof.

### REFERENCES

The following numbered references are cited throughout this disclosure by inclusion of the number reference(s) in square brackets. Each of the following references is hereby incorporated by reference in its entirety.
1. T. A. Ciulla, A. Harris, P. Latkany, H. C. Piper, O. Arend, H. Garzozi and B. Martin, “Ocular perfusion abnormalities in diabetes,” Acta Ophthalmol Scand 80(5), 468-477 (2002)
2. A. Harris, H. S. Chung, T. A. Ciulla and L. Kagemann, “Progress in measurement of ocular blood flow and relevance to our understanding of glaucoma and age-related macular degeneration,” Prog Retin Eye Res 18(5), 669-687 (1999)
3. L. Schmetterer and M. Wolzt, “Ocular blood flow and associated functional deviations in diabetic retinopathy,” Diabetologia 42(4), 387-405 (1999)
4. J. Flammer, S. Orgul, V. P. Costa, N. Orzalesi, G. K. Krieglstein, L. M. Serra, J. P. Renard and E. Stefansson, “The impact of ocular blood flow in glaucoma,” Prog Retin Eye Res 21(4), 359-393 (2002)
5. M. C. Grieshaber, B. Dubler, C. Knodel, H. E. Killer, J. Flammer and S. Orgul, “Retrobulbar blood flow in idiopathic dilated episcleral veins and glaucoma,” Klin Monbl Augenheilkd 224(4), 320-323 (2007)
6. J. C. Hwang, R. Konduru, X. Zhang, O. Tan, B. A. Francis, R. Varma, M. Sehi, D. S. Greenfield, S. R. Sadda and D. Huang, “Relationship among visual field, blood flow, and neural structure measurements in glaucoma,” Invest Ophthalmol Vis Sci 53(6), 3020-3026 (2012)
7. M. T. Nicolela, B. E. Walman, A. R. Buckley and S. M. Drance, “Ocular hypertension and primary open-angle glaucoma: a comparative study of their retrobulbar blood flow velocity,” J Glaucoma 5(5), 308-310 (1996)
8. J. Jonas, M. Paques, J. Mones and A. Glacet-Bernard, “Retinal vein occlusions,” Dev Ophthalmol 47(111-135 (2010)
9. I. Kimura, K. Shinoda, T. Tanino, Y. Ohtake, Y. Mashima and Y. Oguchi, “Scanning laser Doppler flowmeter study of retinal blood flow in macular area of healthy volunteers,” Br J Ophthalmol 87(12), 1469-1473 (2003)
10. M. H. Cuypers, J. S. Kasanardjo and B. C. Polak, “Retinal blood flow changes in diabetic retinopathy measured with the Heidelberg scanning laser Doppler flowmeter,” Graefes Arch Clin Exp Ophthalmol 238(12), 935-941 (2000)
11. M. T. Nicolela, P. Hnik and S. M. Drance, “Scanning laser Doppler flowmeter study of retinal and optic disk blood flow in glaucomatous patients,” Am J Ophthalmol 122(6), 775-783 (1996)
12. R. W. Flower, “Extraction of choriocapillaris hemodynamic data from ICG fluorescence angiograms,” Invest Ophthalmol Vis Sci 34(9), 2720-2729 (1993)
13. M. Venturini, M. Zambon, G. Cristel, G. Agostini, G. Querques, M. Colombo, S. Benussi, G. Landoni, A. Zangrillo and A. Del Maschio, “Monitoring of central retinal artery and vein with color Doppler ultrasound during heart surgery as an alternative to transcranial Doppler ultrasonography: a case report,” J Clin Ultrasound 42(2), 112-115 (2014)
14. P. Sorrentino, G. Tarantino, P. Conca, P. Ragucci and A. Perrella, “Abnormally high resistive index of central retinal artery by ultrasound color Doppler in patients with viral chronic liver disease: correlation with worsening liver staging,” Ultrasound Med Biol 30(5), 599-604 (2004)
15. W. J. Lavery, E. R. Muir, J. W. Kiel and T. Q. Duong, “Magnetic resonance imaging indicates decreased choroidal and retinal blood flow in the DBA/2J mouse model of glaucoma,” Invest Ophthalmol Vis Sci 53(2), 560-564 (2012)
16. Y. Li, H. Cheng, Q. Shen, M. Kim, P. M. Thule, D. E. Olson, M. T. Pardue and T. Q. Duong, “Blood flow magnetic resonance imaging of retinal degeneration,” Invest Ophthalmol Vis Sci 50(4), 1824-1830 (2009)
17. R. Leitgeb, L. Schmetterer, W. Drexler, A. Fercher, R. Zawadzki and T. Bajraszewski, “Real-time assessment of retinal blood flow with ultrafast acquisition by color Doppler Fourier domain optical coherence tomography,” Opt Express 11(23), 3116-3121 (2003)
18. Y. Zhao, Z. Chen, C. Saxer, S. Xiang, J. F. de Boer and J. S. Nelson, “Phase-resolved optical coherence tomography and optical Doppler tomography for imaging blood flow in human skin with fast scanning speed and high velocity sensitivity,” Opt Lett 25(2), 114-116 (2000)
19. X. Li, T. H. Ko and J. G. Fujimoto, “Intraluminal fiber-optic Doppler imaging catheter for structural and functional optical coherence tomography,” Opt Lett 26(23), 1906-1908 (2001)
20. S. Makita, T. Fabritius and Y. Yasuno, “Quantitative retinal-blood flow measurement with three-dimensional vessel geometry determination using ultrahigh-resolution Doppler optical coherence angiography,” Opt Lett 33(8), 836-838 (2008)
21. Y. K. Tao, K. M. Kennedy and J. A. Izatt, “Velocity-resolved 3D retinal microvessel imaging using single-pass flow imaging spectral domain optical coherence tomography,” Opt Express 17(5), 4177-4188 (2009)
22. Y. Wang, B. A. Bower, J. A. Izatt, O. Tan and D. Huang, “In vivo total retinal blood flow measurement by Fourier domain Doppler optical coherence tomography,” J Biomed Opt 12(4), 041215 (2007)
23. Y. Wang, B. A. Bower, J. A. Izatt, O. Tan and D. Huang, “Retinal blood flow measurement by circumpapillary Fourier domain Doppler optical coherence tomography,” J Biomed Opt 13(6), 064003 (2008)
24. Y. Wang, A. A. Fawzi, R. Varma, A. A. Sadun, X. Zhang, O. Tan, J. A. Izatt and D. Huang, “Pilot study of optical coherence tomography measurement of retinal blood flow in retinal and optic nerve diseases,” Invest Ophthalmol Vis Sci 52(2), 840-845 (2011)
25. H. Wehbe, M. Ruggeri, S. Jiao, G. Gregori, C. A. Puliafito and W. Zhao, “Automatic retinal blood flow calculation using spectral domain optical coherence tomography,” Opt Express 15(23), 15193-15206 (2007)
26. O. Tan, Y. Wang, R. K. Konduru, X. Zhang, S. R. Sadda and D. Huang, “Doppler optical coherence tomography of retinal circulation,” J Vis Exp 67), e3524 (2012)
27. Y. Wang, A. Lu, J. Gil-Flamer, O. Tan, J. A. Izatt and D. Huang, “Measurement of total blood flow in the normal human retina using Doppler Fourier-domain optical coherence tomography,” Br J Ophthalmol 93(5), 634-637 (2009)
28. C. Dai, X. Liu, H. F. Zhang, C. A. Puliafito and S. Jiao, “Absolute retinal blood flow measurement with a dual-beam Doppler optical coherence tomography,” Invest Ophthalmol Vis Sci 54(13), 7998-8003 (2013)
29. R. M. Werkmeister, N. Dragostinoff, M. Pircher, E. Gotzinger, C. K. Hitzenberger, R. A. Leitgeb and L. Schmetterer, “Bidirectional Doppler Fourier-domain optical coherence tomography for measurement of absolute flow velocities in human retinal vessels,” Opt Lett 33(24), 2967-2969 (2008)
30. W. Trasischker, R. M. Werkmeister, S. Zotter, B. Baumann, T. Torzicky, M. Pircher and C. K. Hitzenberger, “In vitro and in vivo three-dimensional velocity vector measurement by three-beam spectral-domain Doppler optical coherence tomography,” J Biomed Opt 18(11), 116010 (2013)
31. V. Doblhoff-Dier, L. Schmetterer, W. Vilser, G. Garhofer, M. Groschl, R. A. Leitgeb and R. M. Werkmeister, “Measurement of the total retinal blood flow using dual beam Fourier-domain Doppler optical coherence tomography with orthogonal detection planes,” Biomed Opt Express 5(2), 630-642 (2014)
32. C. Blatter, S. Coquoz, B. Grajciar, A. S. Singh, M. Bonesi, R. M. Werkmeister, L. Schmetterer and R. A. Leitgeb, “Dove prism based rotating dual beam bidirectional Doppler OCT,” Biomed Opt Express 4(7), 1188-1203 (2013)
33. R. M. Werkmeister, N. Dragostinoff, S. Palkovits, R. Told, A. Boltz, R. A. Leitgeb, M. Groschl, G. Garhofer and L. Schmetterer, “Measurement of absolute blood flow velocity and blood flow in the human retina by dual-beam bidirectional Doppler fourier-domain optical coherence tomography,” Invest Ophthalmol Vis Sci 53(10), 6062-6071 (2012)
34. V. J. Srinivasan, D. C. Adler, Y. Chen, I. Gorczynska, R. Huber, J. S. Duker, J. S. Schuman and J. G. Fujimoto, “Ultrahigh-speed optical coherence tomography for three-dimensional and en face imaging of the retina and optic nerve head,” Invest Ophthalmol Vis Sci 49(11), 5103-5110 (2008)
35. W. Choi, B. Baumann, J. J. Liu, A. C. Clermont, E. P. Feener, J. S. Duker and J. G. Fujimoto, “Measurement of pulsatile total blood flow in the human and rat retina with ultrahigh speed spectral/Fourier domain OCT,” Biomed Opt Express 3(5), 1047-1061 (2012)
36. B. Baumann, B. Potsaid, M. F. Kraus, J. J. Liu, D. Huang, J. Hornegger, A. E. Cable, J. S. Duker and J. G. Fujimoto, “Total retinal blood flow measurement with ultrahigh speed swept source/Fourier domain OCT,” Biomed Opt Express 2(6), 1539-1552 (2011)
37. H. C. Hendargo, R. P. McNabb, A. H. Dhalla, N. Shepherd and J. A. Izatt, “Doppler velocity detection limitations in spectrometer-based versus swept-source optical coherence tomography,” Biomed Opt Express 2(8), 2175-2188 (2011)
38. B. Vuong, A. M. Lee, T. W. Luk, C. Sun, S. Lam, P. Lane and V. X. Yang, “High speed, wide velocity dynamic range Doppler optical coherence tomography (Part IV): split spectrum processing in rotary catheter probes,” Opt Express 22(7), 7399-7415 (2014)
39. Y. Jia, O. Tan, J. Tokayer, B. Potsaid, Y. Wang, J. J. Liu, M. F. Kraus, H. Subhash, J. G. Fujimoto, J. Hornegger and D. Huang, “Split-spectrum amplitude-decorrelation angiography with optical coherence tomography,” Opt Express 20(4), 4710-4725 (2012)
40. J. Tokayer, Y. Jia, A. H. Dhalla and D. Huang, “Blood flow velocity quantification using split-spectrum amplitude-decorrelation angiography with optical coherence tomography,” Biomed Opt Express 4(10), 1909-1924 (2013)
41. V. X. D. Yang, M. L. Gordon, A. Mok, Y. Zhao, Z. Chen, R. S. C. Cobbold, B. C. Wilson and I. Alex Vitkin, “Improved phase-resolved optical Doppler tomography using the Kasai velocity estimator and histogram segmentation,” Optics Communications 208(4-6), 209-214 (2002)
42. J. Walther, G. Mueller, H. Morawietz and E. Koch, “Signal power decrease due to fringe washout as an extension of the limited Doppler flow measurement range in spectral domain optical coherence tomography,” J Biomed Opt 15(4), 041511 (2010)
43. T. F. Chan and L. A. Vese, “Active contours without edges,” IEEE Trans Image Process 10(2), 266-277 (2001)
44. J. Strand, T. Taxt and A. K. Jain, “Two-dimensional phase unwrapping using a block least-squares method,” IEEE Trans Image Process 8(3), 375-386 (1999)
45. J. Strand and T. Taxt, “Two-dimensional phase unwrapping using robust derivative estimation and adaptive integration,” IEEE Trans Image Process 11(10), 1192-1200 (2002)
46. D. F. Garway-Heath, A. R. Rudnicka, T. Lowe, P. J. Foster, F. W. Fitzke and R. A. Hitchings, “Measurement of optic disc size: equivalence of methods to correct for ocular magnification,” Br J Ophthalmol 82(6), 643-649 (1998)
47. T. Schmoll and R. A. Leitgeb, “Heart-beat-phase-coherent Doppler optical coherence tomography for measuring pulsatile ocular blood flow,” J Biophotonics 6(3), 275-282 (2013)
48. B. Lee, W. J. Choi, J. J. Liu, C. D. Lu, J. S. Schuman, G. Wollestein, D. Huang, J. S. Duker and J. G. Fujimo, “En fcace Doppler OCT measurement of total reitnal blood flow at 100,000 axial scans per second using pulse oximetry cardiac gating,” in ARVO, p. 5020, Orlando, Fla. (2014).

1. A method of measuring retinal blood flow using optical coherence tomography (OCT), comprising:
receiving an OCT scan dataset of a region of the eye;
generating a reflectance OCT dataset, the reflectance OCT dataset comprised of a set of en face reflectance images;
generating a Doppler OCT dataset, the Doppler OCT dataset comprised of a set of en face Doppler images;
detecting a set of vessels in the set of en face Doppler OCT images;
applying a phase unwrapping algorithm to the set of en face Doppler OCT images;
identifying a set of vein branches in the set of vessels;
determining an optimized en face plane for each of the vein branches; and
calculating a blood flow on the optimized en face plane for each of the vein branches;

2. The method of claim 1, wherein the region of the eye includes the disc boundary of the optic nerve head.

3. The method of claim 1, wherein generating a Doppler OCT dataset is calculated according to Equation (1) and Equation (2).

4. The method of claim 1, further comprising removing bulk motion from the reflectance OCT dataset and the Doppler OCT dataset.

5. The method of claim 1, wherein detecting a set of vessels in an en face Doppler OCT image comprises:
generating a retina mask from the reflectance OCT dataset;
identifying a set of pixels the en face Doppler OCT image having Doppler phase shift values that exceed a threshold, provided that the pixels are located within the retina mask, thereby generating a vessel mask;
removing artifacts from the vessel mask; and
identifying a set of vessels in the en face Doppler OCT image using the vessel mask.

6. The method of claim 5, wherein the threshold is about 2.33 times the standard deviation of Doppler phase shift in a static retinal tissue.

7. The method of claim 1, wherein the optimized en face plane for a vein branch is the depth at which the Doppler phase shift value is maximized.

8. The method of claim 1, wherein calculating a blood flow on the optimized en face plane for a vein branch comprises:
identifying the pixels comprising the vein branch on the optimized en face plane;
transforming the Doppler phase shift values associated with the pixels into speed values;
integrating the speed values over the en face cross sectional area of the vein branch, thereby generating a blood flow; and
correcting the blood flow for eye length variation.

9. The method of claim 1, further comprising calculating a total retinal blood flow (TRBF) by summing the blood flow of each vein branch.

10. The method of claim 9, further comprising calculating a mean TRBF from a plurality of OCT scan datasets.

11. The method of claim 1 further comprising using 70 kHz spectral optical coherence tomography.

12. A method of calculating a Doppler OCT dataset having reduced noise comprising:
receiving a full spectrum OCT dataset;
dividing the full spectrum OCT dataset into a set of narrow band OCT datasets according to Equation 1; and
calculating the Doppler phase shift from the set of narrow band OCT datasets according to Equation 2.
